(19) United States
(12) Patent Application Publication
(10) Pub. No.: US 2018/0161530 A1
(43) Pub. Date: Jun. 14, 2018

(54) FLOW SENSING MEDICINE DELIVERY DEVICE

(71) Applicant: QUALCOMM Incorporated, San Diego, CA (US)

(72) Inventors: Robert Ganton, San Diego, CA (US); Robert Ballam, Eatons Hill (AU); John Earl Amschler, San Diego, CA (US); Paul Nelles, San Diego, CA (US)

(21) Appl. No.: 15/704,999

(22) Filed: Sep. 14, 2017

Related U.S. Application Data
(60) Provisional application No. 62/433,943, filed on Dec. 14, 2016.
(60) Provisional application No. 62/506,496, filed on May 15, 2017.

(51) Int. Cl. A61M 16/00; G01F 1/66; A61M 15/00; A61M 16/12; A61M 16/14

(52) U.S. Cl. CPC A61M 16/0003; A61M 2202/064; A61M 15/0091; A61M 15/0021; A61M 16/12; A61M 16/14; A61M 15/009; A61M 2206/14; A61M 2205/3592; A61M 2205/3375; A61M 2205/3334; A61M 2205/43; A61M 2016/0033; A61M 2202/02; A61M 2202/04; G01F 1/666

(57) ABSTRACT

Methods, systems, computer-readable media, and apparatuses for determining a property of airflow delivered to a user of a medical inhalation device are disclosed.

### CROSS-REFERENCES TO RELATED APPLICATIONS

This patent application claims priority to U.S. Provisional Patent Application Ser. No. 62/433,943, filed Dec. 14, 2016, entitled “FLOW SENSING MEDICINE DELIVERY DEVICE,” and U.S. Provisional Patent Application Ser. No. 62/506,496, filed May 15, 2017, entitled “WIDE BAND FLOW SENSING MEDICINE DELIVERY DEVICE,” which is assigned to the assignee hereof and is incorporated herein by reference in its entirety for all purposes.

### BACKGROUND

Aspects of the disclosure relate to medicine delivering inhalation devices, and more particularly to techniques for determining a quality of delivery of a medicine that a user has inhaled via an inhalation device.

Certain medical devices can be used to deliver a medicine to the lungs of a user via inhalation. One example medical device is an inhalation device than may atomize medicine to, for example, treat chronic obstructive pulmonary disease (COPD) or other ailments when used correctly by a user of the medial device. When used incorrectly, a user may receive an incorrect dosage of medicine that may have adverse effects on the user or may not properly treat an ailment. Furthermore, it may be difficult to verify if a particular user is receiving correct dosage(s) of medicine over time to treat a particular ailment and/or is using an inhalation device properly.

As such, there is need for improvement in the field of medicine delivering inhalation devices.

### BRIEF SUMMARY

Certain embodiments are described that provide techniques for medicine delivery. According to the disclosed techniques, a medical device is provided. The medical device may include a housing that includes a mouthpiece. The medical device may also include a transducer coupled to the housing. The transducer can be configured to generate a signal corresponding to one or more sound waves generated by air flowing towards the mouthpiece. The one or more sound waves can be within an ultrasonic frequency range. The air can include medication for delivery to a user of the medical device. The medical device may further include circuitry coupled to the transducer. The circuitry can be configured to determine, based on the signal, a property corresponding to the air flowing towards the mouthpiece.

In some embodiments, the ultrasonic frequency range may exclude frequencies below twenty kilohertz. The one or more sound waves can include sound waves within at least a one kilohertz frequency range. The medical device can include a physical feature configured to generate the one or more sound waves in response to the air flowing towards the mouthpiece. The physical feature may be configured to divert a portion of the air flowing towards the mouthpiece. The physical feature may further include a knife edge configured to divert the portion of the air flowing towards the mouthpiece in response to the air contacting the knife edge.

In some embodiments, the physical feature of the medical device can be configured to substantially generate the one or more sound waves when the air flows towards the mouthpiece. The physical feature may also be configured not to generate the one or more sound waves when the air flows away from the mouthpiece.

In some embodiments, the physical feature can be configured to generate the one or more sound waves outside of the ultrasonic frequency range. The circuitry of the medical device can be configured to isolate the one or more sound wavers within the ultrasonic frequency range from the one or more sound wave outside of the ultrasonic frequency range.

In some embodiments, the housing of the medical device can be configured to enable the air to flow towards the mouthpiece in response to the user inhaling the air via the mouthpiece. The medical device can be configured to induce the air to flow towards the mouthpiece by pushing the air using positive pressure.

In some embodiments, the medical device can include a switch. The medical device can be configured to introduce the medication into the air in response to an actuation of the switch. The circuitry can be configured to determine the property in response to the actuation of the switch. The transducer can be configured to generate a respective signal corresponding to air flowing towards and away from the mouthpiece. The circuitry can be configured to determine, based on the signals, respective properties corresponding to air flowing towards and away from the mouthpiece. The physical feature can be configured to divert a portion of the air flowing towards the mouthpiece. The physical feature can includes a knife edge configured to divert the portion of the air flowing towards the mouthpiece in response to the air contacting the knife edge.

In some embodiments, the property corresponding to the air flowing towards the mouthpiece and determined by the circuitry may include a rate of flow of the air flowing towards the mouthpiece or a volume of the air flowing towards the mouthpiece.

The medical device can further comprise a medicine receptacle. The medicine included within the air flowing towards the mouthpiece can be inserted into the medical device via the medicine receptacle. The medical device can further comprise a wireless interface coupled to the circuitry. The circuitry can be further configured to transmit, via the wireless interface, data indicative of the property corresponding to the air flowing towards the mouthpiece. The data indicative of the property can include data indicative of a change of the property over time.

According to the disclosed techniques, a method is provided. The method comprises determining, by circuitry coupled to a transducer and based on a signal, a property corresponding to air flowing towards a mouthpiece included within a medical device. The transducer is configured to generate a signal corresponding to one or more sound waves generated by air flowing towards the mouthpiece, wherein the one or more sound waves are within an ultrasonic frequency range and the air includes medication for delivery to a user of the medical device. In some embodiments, the ultrasonic frequency range may exclude frequencies below twenty kilohertz. The one or more sound waves may be within at least a one kilohertz frequency range.

In some embodiments, the one or more sound waves may be generated by a physical feature of the medical device in response to the air flowing towards the mouthpiece. The portion of the air flowing towards the mouthpiece may be diverted by the physical feature. The physical feature may include a knife edge, and the portion of the air flowing towards the mouthpiece is directed by the knife edge in response to the air contacting the knife edge.

In some embodiments, the one or more sound waves are substantially generated when the air flows towards the mouthpiece, and the one or more sound waves are not generated when the air flows away from the mouthpiece. The one or more sound waves may be generated outside of the ultrasonic frequency range. In some embodiments, the method may further comprise isolating, by the circuitry, the one or more sound waves within the ultrasonic frequency range from the one or more sound wave outside of the ultrasonic frequency range.

In some embodiments, the air may be enabled to flow towards the mouthpiece in response to the user inhaling the air via the mouthpiece. The air may also be induced to flow towards the mouthpiece by a positive pressure.

In some embodiments, the method may further comprise determining the property in response to an actuation of a switch included in the medical device. The medication can be introduced into the air in response to the actuation of the switch.

In some embodiments, a respective signal corresponding to the one or more sound waves is generated in response to air flowing, respectively, towards and away from the mouthpiece. The method may further comprise: determining, by the circuitry and based on the respective signals corresponding to the one or more sound waves generated in response to air flowing, respectively, towards and away from the mouthpiece, respective properties corresponding to the air flowing towards and away from the mouthpiece.

In some embodiments, the property includes a rate of flow of the air flowing towards the mouthpiece or a volume of the air flowing towards the mouthpiece.

In some embodiments, the medical device may further comprise a medicine receptacle. The medicine included within the air flowing towards the mouthpiece may be inserted into the medical device via the medicine receptacle.

In some embodiments, the medical device further comprises a wireless interface coupled to the circuitry; wherein the method further comprises: transmitting, via the wireless interface, data indicative of the property corresponding to the air flowing towards the mouthpiece. The data indicative of the property may include data indicative of a change of the property over time.

According to the disclosed techniques, an medical apparatus is provided. The apparatus comprises a means for generating a signal corresponding to one or more sound waves generated by air flowing towards a mouthpiece of a medical apparatus, wherein the one or more sound waves are within an ultrasonic frequency range and the air includes medication for delivery to a user of the medical apparatus. The medical apparatus further comprises a means for determining, based on the signal, a property corresponding to the air flowing towards the mouthpiece.

In some embodiments, the medical apparatus further comprises a means for generating the one or more sound waves in response to the air flowing towards the mouthpiece. The means for generating the one or more sound waves in response to the air flowing towards the mouthpiece may comprise a means for diverting a portion of the air flowing towards the mouthpiece. The means for generating the one or more sound waves in response to the air flowing towards the mouthpiece may also comprise a means for generating one or more sound waves outside of the ultrasonic frequency range. The medical apparatus may further comprise a means for isolating the one or more sound waves within the ultrasonic frequency range from the one or more sound wave outside of the ultrasonic frequency range.

In some embodiments, the medical apparatus may further comprise a means for enabling the air to flow towards the mouthpiece in response to the user inhaling the air via the mouthpiece, or for inducing the air to flow towards the mouthpiece using positive pressure. The medical apparatus may further comprise a means for triggering an introduction of the medication into the air; and a means for determining the property in response to an actuation of the means for triggering the introduction of the medication into the air.

In some embodiments, the medical apparatus may further comprise a means for generating a respective signal corresponding to the one or more sound waves generated in response to air flowing, respectively, towards and away from the mouthpiece; and a means for determining, based on the respective signals, respective properties corresponding to the air flowing, respectively, towards and away from the mouthpiece. The medical apparatus may further comprise a means for transmitting, wirelessly, data indicative of the property corresponding to the air flowing towards the mouthpiece, wherein the data indicative of the property includes data indicative of a change of the property over time.

### BRIEF DESCRIPTION OF THE DRAWINGS

Aspects of the disclosure are illustrated by way of example. In the accompanying figures, like reference numbers indicate similar elements.

FIG. 1 illustrates a simplified diagram of a system that may incorporate one or more features of the disclosure pertaining to an inhalation device communicatively coupled to a mobile device;

FIG. 2 illustrates a simplified diagram of a system that may incorporate one or more disclosed features of an inhalation device;

FIG. 3 illustrates a simplified diagram of a use case of certain embodiments of disclosed inhalation devices;

FIG. 4 illustrates a simplified diagram of an additional use case of certain embodiments of disclosed inhalation devices;

FIG. 5 illustrates a simplified diagram of certain audio spectrum related features of the disclosure pertaining to inhalation devices;

FIG. 6 illustrates an example flowchart of a method of use of certain disclosed features; and

FIG. 7 illustrates an example of a computing system in which one or more embodiments may be implemented.

### DETAILED DESCRIPTION

Several illustrative embodiments will now be described with respect to the accompanying drawings, which form a part hereof. While particular embodiments, in which one or more aspects of the disclosure may be implemented, are described below, other embodiments may be used and various modifications may be made without departing from the scope of the disclosure or the spirit of the appended claims.

Medicine delivering inhalation devices can accept liquid, gaseous, powdered, or other forms of medication for delivery to a user's lungs when a user inhales the medicine while using the inhalation device. The user may also inhale a fluid, such as air, that may act as a carrier and/or aid in dispersal of the medicine to lungs of the user. Air, as utilized herein, can be any fluid that can be inhaled by a user. Examples can include nitrogen and/or oxygen. The inhalation device can include features that atomize, particularize, or otherwise disperse the medicine so that it is properly distributed into the air.

Disclosed inhalation devices can receive medicine in several different ways prior to deliver of the medicine to a user. For example, an inhalation device can include a port to accept a removable cartridge containing medicine. In some examples, the port may also accept the medicine in other forms (such as in liquid form) and/or from different containers. In certain examples, an inhalation device may be disposable and/or the port for receiving medicine can be integrated within a body of the device. In certain embodiments, medicine can be delivered via a compressed fluid/air. A fluid flow for delivery of medicine can be induced by a user manually inhaling in proximity to or contact with an inhalation device, creating a relatively low pressure area within lungs of the user. The low pressure area can induce flow of air and medicine from an inhalation device to lungs of the user.

In practice, a user of an inhalation device may not receive an effective dosage and/or consistent dosage(s) of medicine via the inhalation device for several reasons. For example, a user may have unknowingly exhausted the medicine of an inhalation device. A user may not properly seat medicine (or a medicine cartridge) within a device, not inhale properly, forget dosage(s), accidentally receive too many dosages, misunderstand dosage instructions, incorrectly operate the inhalation device, have been prescribed an incorrect dosage, etc. Therefore, many difficulties may persist when verifying that user(s) are being correctly medicated via inhalation devices. Furthermore, it may be difficult to ascertain results of certain dosage pattern(s) with respect to a large population set of users.

Disclosed are techniques to determine a property of airflow within an inhalation device for delivery of medicine to a user of the inhalation device. The techniques can include use of an inhalation device that can include a sensor to determine a magnitude of fluid flow or volume of fluid inhaled by or otherwise delivered to lungs of a user. The sensor can detect an audio signature created by air flowing through the medical device. For example, the device can be configured with one or more features that are configured to generate audio waves in response to air flowing through the device to lungs of a user. The audio waves can be generated over a relatively broad frequency range which may include human audible audio waves (generally in a frequency range of twenty hertz to twenty kilohertz) and/or ultrasonic frequency ranges (generally in a frequency range above twenty kilohertz). In certain embodiments, non-human audible frequency range(s) of audio can be analyzed in order to avoid audible noise within a user's environment that may interfere with measurements and/or be a nuisance to the user.

The device can include circuitry, such as a controller or processor, which can be configured to determine one or more properties of the air flow based on analyzing an audio signature generated by the sensor corresponding to air flowing to the lungs of a user. The one or more properties can be used to determine a magnitude of fluid flow over time, a magnitude of medicine introduced via the fluid to a user, a total dosage delivered to the user (e.g., a magnitude of medicine over time), or a combination of the preceding. Determining the magnitude of fluid flow or medicine can include determining one or more corresponding properties of audio waves generated by air traveling towards lungs of a user. For example, an amount of energy of the audio waves can be correlated to a magnitude of fluid flow.

In certain embodiments, an ultrasonic frequency range of audio can be examined to, for example, provide noise immunity to interference caused by audio waves within an audible frequency range. A relatively broad frequency range may be examined to, for example, provide immunity from manufacturing variations of physical features of an inhalation device to configured to generate audio wave(s) in response to fluid flowing in contact with the physical features. As disclosed herein, a medical device, such as an inhaler, can be manufactured including features of the disclosure to determine a fluid flow/an amount of medicine delivered to a user. Price may be a concern for manufacture of such devices. Specifying relatively high manufacturing tolerances for such a device may increase its cost for manufacture. Relatively low manufacturing tolerances of such a device may correspond to a device that produces audio across a relatively wide frequency range and/or at relatively highly variable frequencies. By utilizing a relatively large audio frequency range when determining a magnitude of fluid or medicine flow, such variability can be accounted for while still enabling a relatively accurate magnitude to be determined.

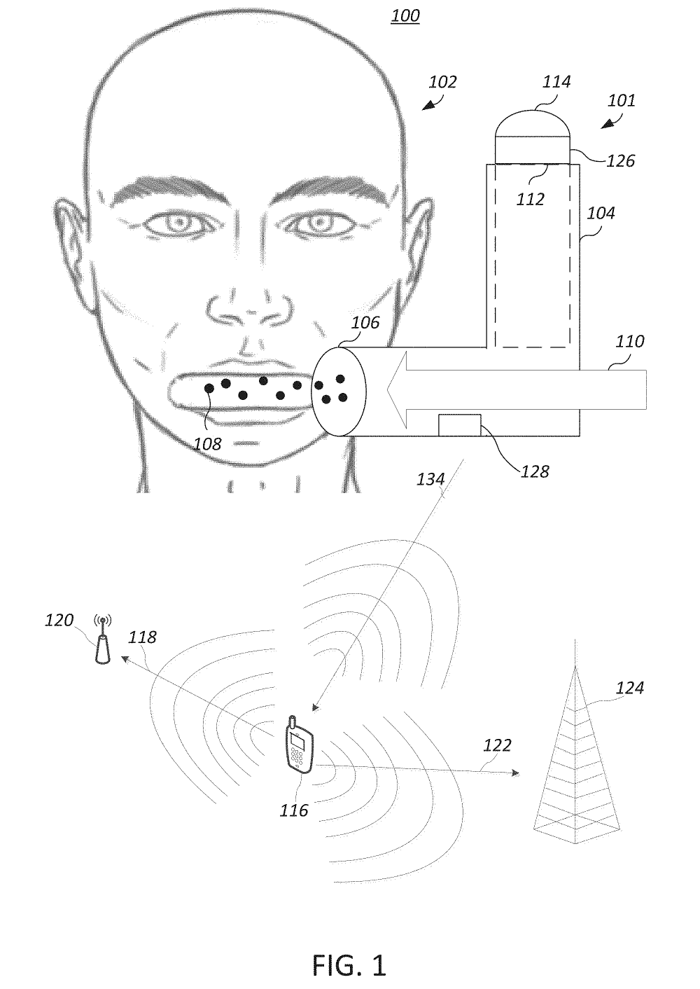

FIG. 1 illustrates a simplified diagram of an inhalation system 100 including a medical inhalation device 101 that can be an inhaler or similar medical device to atomize medicine for delivery to a user, such as user 102. User 102 of medical inhalation device 101 can receive medicine 108 via a port 106 of medical inhalation device 101. Port 106 can include a mouthpiece for interfacing with a mouth of user 102. Port 106 may also or alternatively include a nostril piece for coupling with nostril(s) of user 102. User 102 can, for example, inhale medicine 108 from port 106 and/or medicine 108 can be pressurized and forced, using positive pressure delivery mechanism(s), into a mouth of user 102 in a direction indicated by arrow 110. Medical inhalation device 101 can include a port 112 to accept a medicine container 126. Ports 106 and 112 can be in fluid communication with each other and can be defined by housing 104 of medical inhalation device 101. In certain embodiments, medical inhalation device 101 can include a switch 114 which can initiate delivery of medicine 108 to user 102 and/or initialize data monitoring/collection.

Medical inhalation device 101 can include circuitry 128 to collect information pertaining to delivery of medicine 108 to user 102. Switch 114 can be used, for example, to initialize circuitry 128 or induce circuitry 128 to enter a higher power state and/or gather sensor data. Circuitry 128 can be coupled to a sensor (not shown) to determine a magnitude of flow of fluid and/or medicine that is delivered to user 102. Circuitry 128 can sample a magnitude of fluid flow over time. An amount of medicine delivered by actuation of switch 114 over time can also be recorded and/or determined by circuitry 128. By comparing magnitudes of medicine delivery and/or magnitude(s) of air flow, circuitry 128 can determine how much medicine 108 has been delivered to user 102 at different time(s). Circuitry 128 can also be coupled to sensor(s) (not illustrated) to determine whether medical inhalation device 101 is operating correctly, and/or whether medical inhalation device 101 is being operated correctly by user 102. For example, sensor(s) can determine information about an orientation of medical inhalation device 101, information indicating whether switch 114 is correctly actuated, information indicating whether user 102 inhales at the proper time when medicine 108 is dispersed, information indicating whether medicine container 126 is properly seated, etc. Circuitry 128 can perform one or more transformations on data depending on one or more calibration profiles, for example, contained therein. The profiles and/or functionality of circuitry 128 can be approved, validated, and/or configuration controlled in accordance to food and drug administration (FDA) regulations, for example.

Medical inhalation device 101, although not illustrated, can include a power source to power circuitry 128, for example. The power source can be hermetically sealed to mitigate danger to user 102. The power source can include a battery and/or capacitor(s), for example. The power source can include a power source or being wirelessly charged (such as via inductive charging).

Circuitry 128 can be coupled to or include a transceiver to enable wireless communication with mobile device 116. Mobile device 116 may be a device designed to perform numerous functions, including the ability to communicate, via a relatively long distance communication link, with a server (not shown). Mobile device 116 can be, for example, a smartphone, swart watch, or tablet, for example, that may include a Bluetooth® communication link with medical inhalation device 101 and/or a cellular link to communicate with the server. The server can collect dosage information from various users of inhalation devices (such as user 102) to determine usage patterns, provide calibration profile(s), verify correct dosage delivery, and/or verify correct usage of medical inhalation device 101, for example.

In the example shown in FIG. 1, mobile device 116 can wirelessly communicate by sending signals to, and receiving signals from, one or more access points 120. For instance, mobile device 116 may send a communication signal 118 to an access point 120, which may be a base station supporting cellular or WiFi® communications, for example. Mobile device 116 may also or alternatively send a communication signal 122 to cell tower 124, which may be a base station supporting cellular communications. Communication signal 134 between inhalation device 101 and mobile device 116 can be a relatively lower power signal and/or have limited range as compared to communication signals 118 an/or 122. In certain embodiments, medical inhalation device 101 may be constrained with regards to cost, operating time requirement(s), weight, and/or size and therefore power usage and/or battery weight necessary to support longer range communications may not be integrated into a medical device used within medical inhalation device 101. Privacy concerns may also dictate use of a relatively limited range and/or low power signal for communicating between system and mobile device 116.

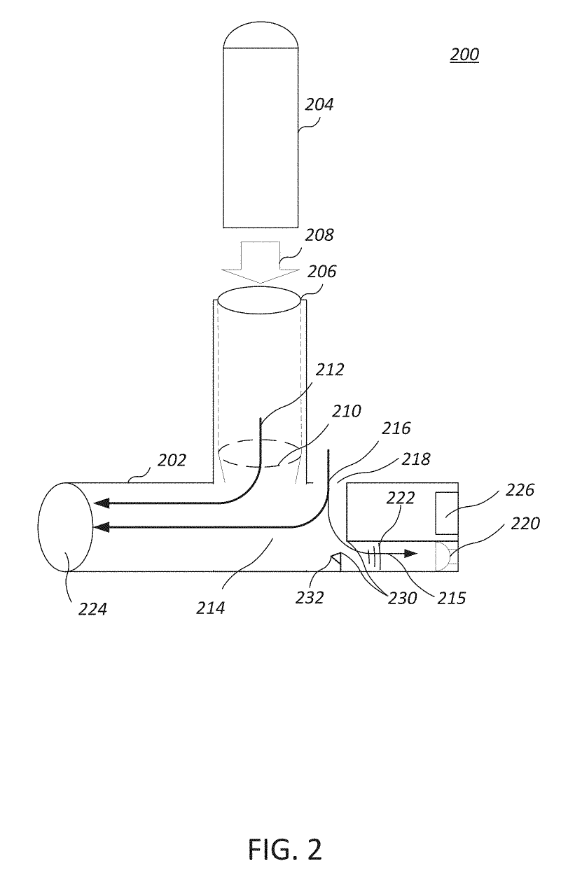

FIG. 2 illustrates an exemplary embodiment of an inhalation system 200. The system 200 can include an inhalation device housing 202 that can be similar to housing 104. Housing 202 can include a port 206 to receive a medicine container 204 via a direction indicated by an arrow 208. Medicine container 204 can contact an internal port 210 of device housing 202. When a user presses downward in direction of arrow 208 on medicine container 204 after medicine container 204 is inserted into housing 202, medicine contained therein may be dispersed into a cavity defined by housing 202. Pressing of downward in direction of arrow 208 may also activate a switch, such as switch 114.

Housing 202 can include a port 218 to, for example, allow atmospheric air to enter into cavity 214 of housing 202. Although not illustrated, port 210 and/or 218 can be integrated as a single port. In certain embodiments, airflow can enter through cavity 214 from a medicine container 204 (which may by pressurized, for example). Cavity 214 can include one or more physical features 230. Features 230 can be configured to generate audio waves within a non-audible frequency range and/or over a relatively broad frequency range (for example, over a one kilohertz frequency range) when a user inhales via port 224. The frequency range can include human audible and/or inaudible frequency spectrums. Human audible frequency ranges can include frequencies of audio in the range of twenty Hertz to twenty kilohertz.

When a user inhales via port 224, atmospheric air can be induced to flow through a path indicated by arrow 216. A portion of the air traveling through path indicated by arrow 216 can be diverted by features 230 in a directed indicated by arrow 215 as illustrated in FIG. 2. Features 230 can, as illustrated, form a relatively small opening for air to flow through. The design of features 230 can induce sound wave(s) 222 to be emitted when air flows in the direction indicated by arrows 215 (and 216).

Features 230 can, for example, include an edge 232 over which air or another fluid flows (such as air flow occurring through path illustrated by arrow 215). The Edge 232 can be a sharp edge as illustrated, a rounded edge, or other. As the air flows over features 230, a pressure wave or turbulence can be induced that results in the generation of sound waves 222. A transducer 220 can be coupled to housing 202 to act as a microphone and convert sound/pressure waves 222 into electrical signals that can be processed by circuitry 226. Transducer 220 can be configured to respond to a certain frequency range of sound 222 (e.g., an ultrasonic frequency range and/or a human audible frequency range). In certain embodiments, circuitry 226 can be configured to filter certain frequencies of audio. For example, audible frequency ranges may be filtered out of a signal generated by transducer 220. In certain embodiments, transducer 220 can be configured to generate a signal in response to non-audible sound waves being incident upon transducer 220. Transducer 220 can also be configured not to generate the signal in response to audible sound waves being incident upon transducer 220. By filtering or isolating audible sound waves from a corresponding signal, sound waves 222 corresponding to fluid flow through housing 202 can be more easily differentiated from background audible noise. By analyzing one or more properties of sound waves 222 or a signal generated in response to sound waves 222 (e.g., an amount of energy contained within a signal waveform, an amount of energy over time, an amplitude, a shape of a waveform, a peak of a waveform, etc.), a magnitude of airflow traveling through housing 202 can be determined. Circuitry 226 can determine, for example, a peak flow rate, a total inhaled volume, and/or a total medical dosage over a certain time period.

Airflow traveling through housing 202 in direction indicated by arrow 216 can interact with medicine that is dispersed into housing 202. The medicine can follow a path indicated by arrow 212 out through port 224 and to user 102 of FIG. 1. Circuitry 226 can be similar to circuitry 128 and can include functionality to, for example, determine magnitude of medicine delivered/airflow vs time, communication with a mobile device, etc.

System 200 is provided as an example. The components and/or relative size of components can vary without deviating from the disclosure. For example, features 230 can be manufactured at any dimension or at any position to induce sound 222 to be generated as a result of air traveling through housing 202. Edge 232 can be one of several edges that can be dispersed in several location(s) within system 200. System 200 can be configured to record a magnitude of a fluid flow that does not include medicine. For example, sound 222 may be generated as a results of fluid prior to introduction of medicine.

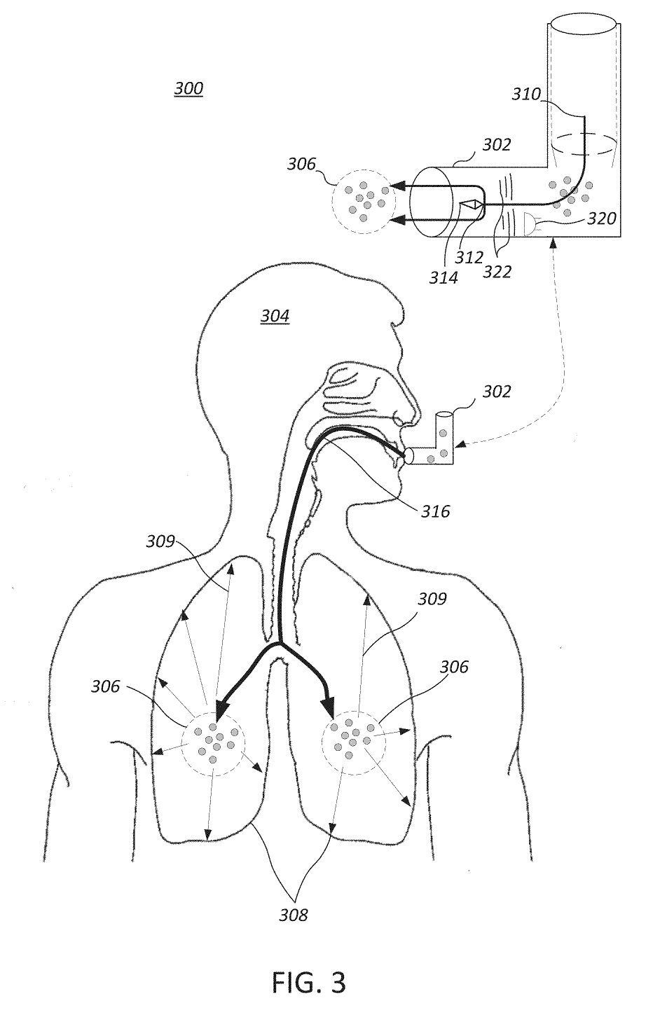

FIG. 3 illustrates a use case 300 of a medical inhalation device 302 that can be similar to system 200 of FIG. 2. Medical inhalation device 302 can deliver medicine 306 to user 304 via inhalation. As disclosed herein, medicine 306 can be atomized and delivered via a fluid flow to lungs 308 of user 304. As user 304 inhales, lungs 308 can expand, as illustrated by corresponding arrows 309. As lungs 308 expand, a relatively low pressure area can be created within lungs 308 that can induce fluid (such as air containing medicine) to be drawn into lungs 308 from medical inhalation device 302 (as illustrated by arrows 316 and 310). Medicine 306 can then be absorbed and/or otherwise interact with lungs 308 of user 304 to treat ailments.

When air is inhaled by user 304 in a direction indicated by arrow 316, air may also flow within medical inhalation device 302 in a direction indicated by arrow 310. Features 312 and 314 can be similar to features 230 of system 200 and can be used to generate audio waves 322 in response to air flow in direction of arrow 310. Furthermore, a transducer 320 can be configured to detect sound waves 322 and generate a corresponding signal to be analyzed by corresponding circuitry (not illustrated, but described with respect to FIGS. 1 and 2) to determine a magnitude of a property of airflow in the direction of arrow 310.

Determining the magnitude of the property can include, for example, an integration or otherwise a summation of an amplitude signal over time generated by transducer 320 in response to air flowing in direction of arrow 310. Depending on a certain type of medicine and/or device 302, an expected density ratio of medicine 306 to air may be known when the medicine is delivered to a user. The expected density ratio can be used to determine an amount of medicine 306 delivered to user 304. The ratio may be static or may change over time during a dosage, for example. Thus, a particular calibration curve can be used to determine a specific amount of medicine delivered to user 304 during a dosage. The calibration curve can be time sensitive depending on an actuation of a switch, such as switch 114, and/or various other variables. For example, a temperature, humidity, altitude, or similar environmental factors can be used for a certain calibration curve. In certain embodiments, a calibration curve can be implemented by a mobile device, such as mobile device 116, and/or by circuitry within an inhalation device/system (such as medical inhalation device 302). Calibration profiles can be loaded by a server coupled to mobile device 116, for example, depending upon a type of inhalation device, a medicine used with an inhalation device, environmental factors, etc. Environmental factors can be determined based on geographical location information of user 304, for example, and/or sensed via sensor(s) of mobile device 116 or medical inhalation device 302. A usage time or medicine usage of device 302 or a medicine storage container coupled to device 302 can be used as input to a calibration curve in certain embodiments. For example, if device 302 has a relatively new medicine storage container or has been freshly refilled with medicine, then airflow may contain more medicine per unit time as compared to a nearly empty container/device.

In certain embodiments, an amount of medicine need not be determined. A magnitude of a property of airflow in direction of arrow 310 can be used to determine, without determining an amount of medicine, whether a user is obtaining a proper dosage of medicine 306. For example, an amount of air inhaled by user 304 can be used to determine whether user 304 has received a quality dosage of medicine 306 and/or received medicine 306 at proper times/intervals. In certain embodiments, a property of airflow can indicate whether user 304 is properly utilizing medical inhalation device 302. For example, a tone or other property of sound waves 322 can be analyzed to determine whether user 304 has formed a proper seal with device 302 so that medicine is delivered to lungs 308 instead of the atmosphere, for example. Although not illustrated, sensor(s) of medical inhalation device 302 can be used to determine an orientation of device 302, proper sealing between user 304 and medical inhalation device 302, and/or other readings indicative of proper usage of medical inhalation device 302. Sensors can include gyroscope(s), magnetometer(s), contact sensor(s), infrared sensor(s), etc.

A magnitude of a property of airflow in direction of arrow 310 can be also be logged by medical inhalation device 302, mobile device 116, or a server coupled to medical inhalation device 302 or mobile device 116. Such information can be correlated over multiple users and can be analyzed depending on several factors to determine proper dosage and/or usage information, for example. For example, dosage information corresponding to users of certain ages, weights, heights, locations, environmental conditions, etc. can be collated and analyzed to determine calibration profile(s), usage statistics, or other information pertinent to delivery of medicine to user(s) of medical inhalation device 302. Thus, medical inhalation device 302 can be used as a data mining tool as well as or instead of a verification tool.

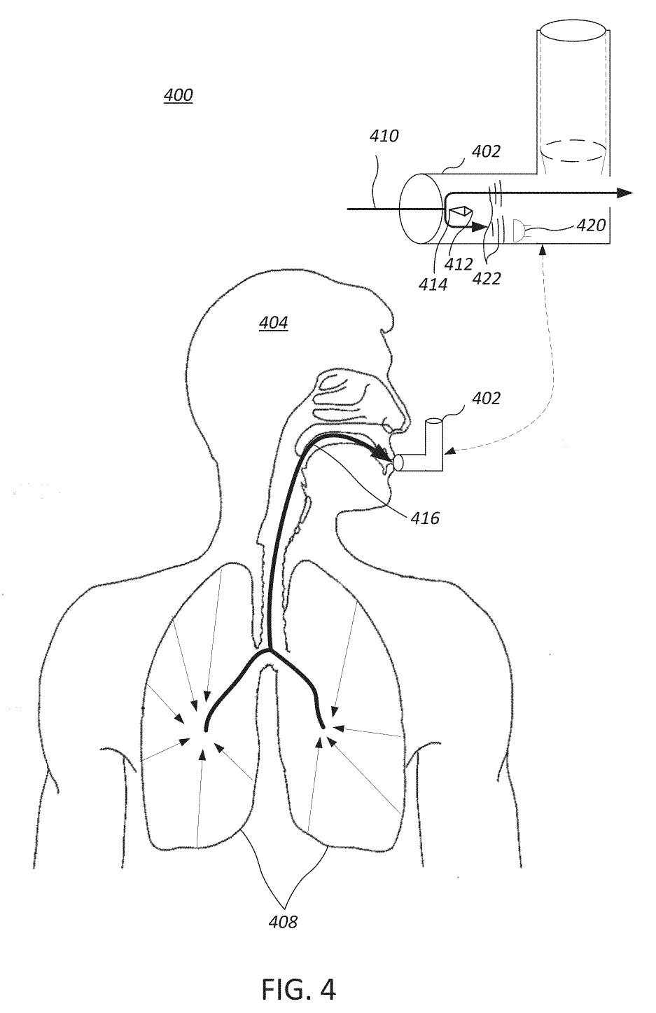

FIG. 4 illustrates another use case of disclosed medical inhalation device(s). Illustrated is system 400 that can include a medical inhalation device 402 which can be similar to medical inhalation device 302. User 404 of medical inhalation device 402 is illustrated as exhaling, inducing air to flow in a direction indicated by arrow 416. When exhaling, lungs 408 of user can contract, creating a relatively high pressure area within lungs 408, forcing air outwards through positive displacement.

When user 404 inhales through medical inhalation device 402, air flowing in direction of arrow 416 can also flow in direction of arrow 410 or induce flow of air in diction of arrow 410. Medical inhalation device 402 can include feature 414 in addition to feature 412 (that can be similar to feature 312). Feature 414 can include sound waves 422 to be generated in response to air flowing in direction of arrow 410. Sound waves 422 may be different than sound waves 322. For example, sound waves 422 may operate within a different frequency range, have a different tone, for a different tonal or amplitude pattern, etc. Sound waves 422 may operate within a non-audible frequency range.

Transducer 420 may be similar to transducer 320 of FIG. 3 and may generate a corresponding signal in response to sound waves 422 being incident upon transducer 420. The signal can be analyzed by circuitry (such as circuitry 128, for example), to determine one or more properties of air flowing in direction of arrow 410. The properties can be analyzed to determine whether user 404 is properly using medical inhalation device 402, for example. For example, a user may be required to, for a proper dosage, inhaled and exhale in succession or a certain number of times for a proper dosage of medicine to be delivered to lungs 408 of user 404.

In certain embodiments, medical inhalation device 402 can operate as a spirometer to measure how much air flows in direction arrow 416 (and thus 410) as a user exhales. Such information can be used to gauge the health of lungs 408 of user 404. By assessing a quality of inhalation and/or exhalation of a user over time, effectiveness of medicine delivered by medical inhalation device 402 to user 404 can be assessed. For example, a determination of an improvement or degradation of the health of lungs 408 can be made by measuring an amount of air inhaled or exhaled by user and/or an amount of air inhaled or exhaled over time. Such information can be used for data mining purposes and/or for verification of dosage(s) delivered to a user, for example. In certain embodiments medical inhalation device 402 can be used solely as a spirometer without medicine delivery capabilities.

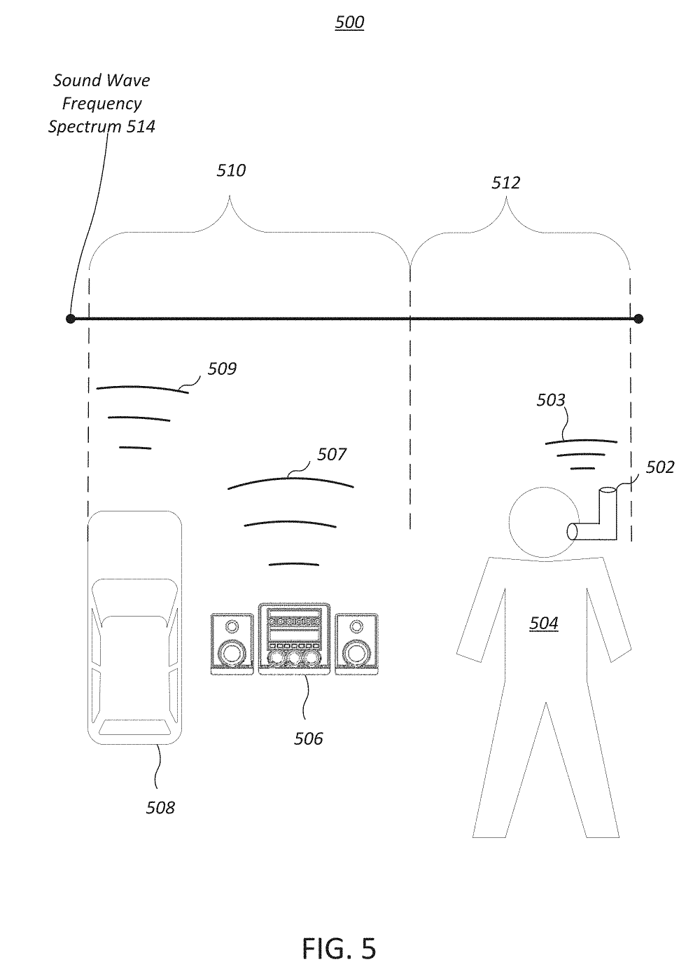

FIG. 5 illustrates a simplified diagram of a system 500 illustrating certain features of the disclosure. FIG. 5 illustrates medical inhalation device 502, which can be similar to medical inhalation device 402 and user 504 which can be similar to user 404. Also illustrated is a sound wave frequency spectrum 514 with frequency ranges 510 and 512 designated. As illustrated, user 504 may be in proximity to various noise sources that may operate at audible frequencies. For example, a car 508 is illustrated that may generate sound waves 509. Sound waves 509 can include a horn, exhaust note, car stereo, etc. Stereo 506 is also illustrated that may generate corresponding sound waves 507. Sound waves 507 can be music for example.

Although only car 508 and stereo 506 are illustrated as examples, it should be understood that user 504 may be in contact with a vast number of variety of audible noise including animals, machines, other people, etc. Medical inhalation device 502 can operate by examining sound waves 503 generated in response to air being exhaled or inhaled by user 504. Sound waves 503 may be difficult to analyze to determine properties of the air flow because of noise pollution induced by audible environmental noise in proximity to user 504.

Techniques of the disclosure can mitigate noise generated by audible noise generators by, for example, analyzing or generating sound waves 503 within a non-audible frequency range that may avoid audible frequency noise. As illustrated, audible noise (such as sound waves 509 and 507) can fall within an audible range of frequency range 510. Sound waves 503 can fall within non-audible frequency range 512 to avoid interference by sound waves 507 or 509, for example. In certain embodiments, frequency range 512 can be an ultrasonic frequency range above approximately twenty kilohertz. In certain embodiments, sound waves 503 may fall within frequency ranges 510 and 512 but only a portion within frequency range 512 may be analyzed. In certain embodiments, a substantial portion of sound waves 503 may fall within a non-audible frequency spectrum or certain impulses of sound waves 503 may fall within a non-audible frequency spectrum for further analysis.

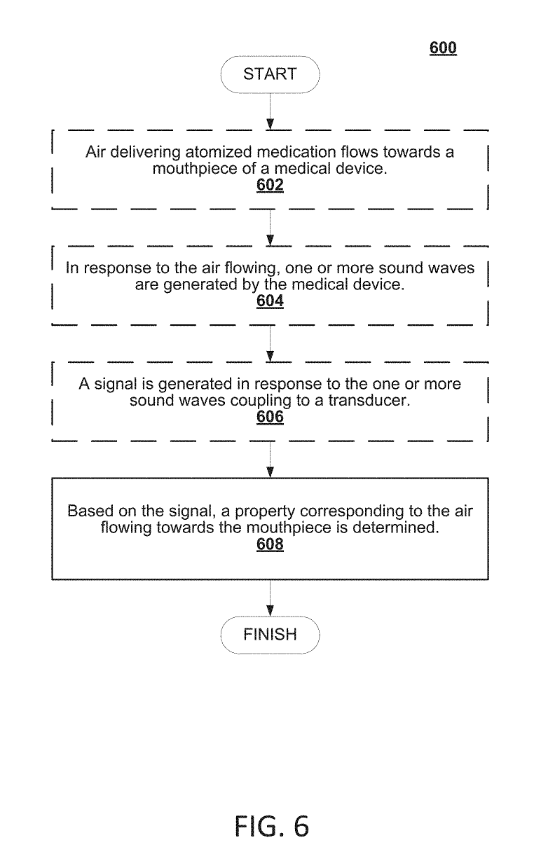

FIG. 6 illustrates a flowchart 600 that can be used to implement certain features of the disclosure corresponding to a medical inhalation device 402, such as medical inhalation device 502. At 602, air delivering atomized medication can flow towards a mouthpiece of a medical device (such as a medical inhalation device). The medicine can be a solid (powder) or liquid as disclosed herein. The mouthpiece can include or instead be configured to be coupled to a nose of a user. The air can be induced by a user inhaling or by positively pressurizing the air to push it out of the device.

At 604, in response to the air flowing, one or more sound wave can be generated by the medical device. For example, features 230 and/or 312 can be used to generate corresponding sound waves in response to air flow in contact with the features. The features can include a knife edge, resonator, resonant cavity, etc. and may induce sound waves to be generated over a relatively large or varied frequency range (such as over at least approximately one kilohertz) and/or within a non-audible frequency range.

At 606, a signal can be generated in response to the one or more sound waves coupling to the transducer. The transducer can be transducer 220, 320 or 420 for example. The transducer can include a magnetic element that moves in response to incident pressure/sound waves, inducing a corresponding electrical signal to be generated. At 608, based on the signal, a property corresponding to the air flowing towards the mouthpiece can be determined. The property can be magnitude of air flow, a direction of air flow, a magnitude of air flow over time. One or more quality factors may also be determined based on the signal and/or additional sensor signal(s). The signals can be analyzed by a circuit of the medical device, a coupled mobile device, and/or a coupled server that may be coupled to multiple medical devices and/or mobile devices. Using the property can be used to verify dosage of medicine to a user, assess user improvement/health, and/or for data mining purposes as disclosed herein.

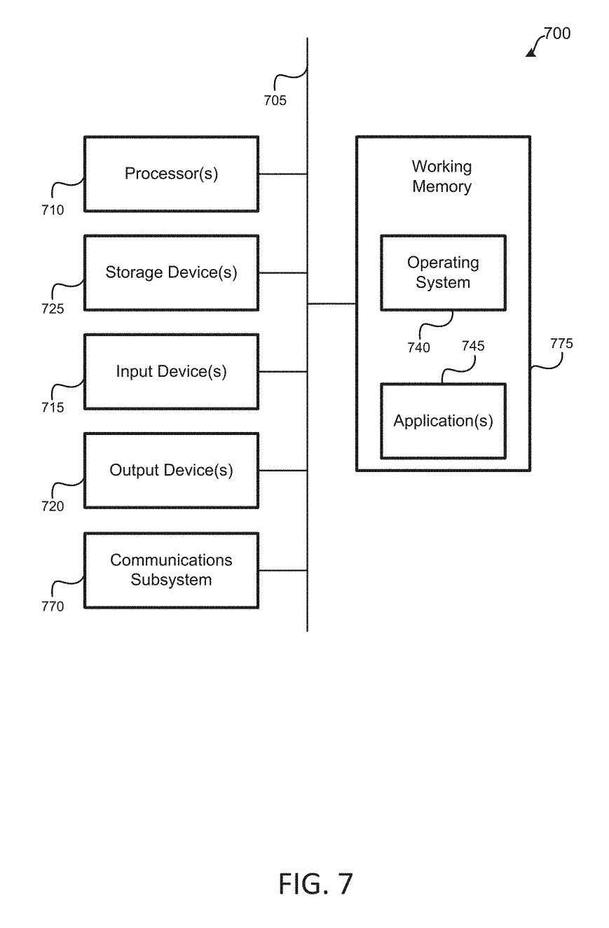

FIG. 7 provides a schematic illustration of one embodiment of a computer system 700 that can perform various blocks of the methods provided by various embodiments. A computer system as illustrated in FIG. 7 may be incorporated as part of the previously described computerized devices, such as mobile device 116, circuitry 128, circuitry 226, etc. It should be noted that FIG. 7 is meant only to provide a generalized illustration of various components, any or all of which may be utilized as appropriate. FIG. 7, therefore, broadly illustrates how individual system elements may be implemented in a relatively separated or relatively more integrated manner.

The computer system 700 is shown comprising hardware elements that can be electrically coupled via a bus 705 (or may otherwise be in communication, as appropriate). The hardware elements may include one or more processors 710, including without limitation one or more general-purpose processors and/or one or more special-purpose processors (such as digital signal processing chips, graphics acceleration processors, video decoders, and/or the like); one or more input devices 715, which can include without limitation a mouse, a keyboard, remote control, and/or the like; and one or more output devices 720, which can include without limitation a display device, a printer, and/or the like. As used herein, a controller can include functionality of a processor (such as processors 710).

The computer system 700 may further include (and/or be in communication with) one or more non-transitory storage devices 725, which can comprise, without limitation, local and/or network accessible storage, and/or can include, without limitation, a disk drive, a drive array, an optical storage device, a solid-state storage device, such as a random access memory (“RAM”), and/or a read-only memory (“ROM”), which can be programmable, flash-updateable and/or the like. Such storage devices may be configured to implement any appropriate data stores, including without limitation, various file systems, database structures, and/or the like.

The computer system 700 might also include a communications subsystem 770, which can include without limitation a modem, a network card (wireless or wired), an infrared communication device, a wireless communication device, and/or a chipset (such as a Bluetooth™ device, an 802.11 device, a Wi-Fi device, a WiMax device, cellular communication device, GSM, CDMA, WCDMA, LTE, LTE-A, LTE-U, etc.), and/or the like. The communications subsystem 770 may permit data to be exchanged with a network (such as the network described below, to name one example), other computer systems, and/or any other devices described herein. In many embodiments, the computer system 700 will further comprise a working memory 775, which can include a RAM or ROM device, as described above.

The computer system 700 also can comprise software elements, shown as being currently located within the working memory 775, including an operating system 740, device drivers, executable libraries, and/or other code, such as one or more application programs 745, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a non-transitory computer-readable storage medium, such as the non-transitory storage device(s) 725 described above. In some cases, the storage medium might be incorporated within a computer system, such as computer system 700. In other embodiments, the storage medium might be separate from a computer system (e.g., a removable medium, such as a compact disc), and/or provided in an installation package, such that the storage medium can be used to program, configure, and/or adapt a general purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer system (such as the computer system 700) to perform methods in accordance with various embodiments of the invention. According to a set of embodiments, some or all of the procedures of such methods are performed by the computer system 700 in response to processor 710 executing one or more sequences of one or more instructions (which might be incorporated into the operating system 740 and/or other code, such as an application program 745) contained in the working memory 775. Such instructions may be read into the working memory 775 from another computer-readable medium, such as one or more of the non-transitory storage device(s) 725. Merely by way of example, execution of the sequences of instructions contained in the working memory 775 might cause the processor(s) 710 to perform one or more procedures of the methods described herein.

The terms “machine-readable medium,” “computer-readable storage medium” and “computer-readable medium,” as used herein, refer to any medium that participates in providing data that causes a machine to operate in a specific fashion. These mediums may be non-transitory. In an embodiment implemented using the computer system 700, various computer-readable media might be involved in providing instructions/code to processor(s) 710 for execution and/or might be used to store and/or carry such instructions/code. In many implementations, a computer-readable medium is a physical and/or tangible storage medium. Such a medium may take the form of a non-volatile media or volatile media. Non-volatile media include, for example, optical and/or magnetic disks, such as the non-transitory storage device(s) 725. Volatile media include, without limitation, dynamic memory, such as the working memory 775.

Common forms of physical and/or tangible computer-readable media include, for example, a floppy disk, a flexible disk, hard disk, magnetic tape, or any other magnetic medium, a CD-ROM, any other optical medium, any other physical medium with patterns of marks, a RAM, a PROM, EPROM, a FLASH-EPROM, any other memory chip or cartridge, or any other medium from which a computer can read instructions and/or code.

Various forms of computer-readable media may be involved in carrying one or more sequences of one or more instructions to the processor(s) 710 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 700.

The communications subsystem 770 (and/or components thereof) generally will receive signals, and the bus 705 then might carry the signals (and/or the data, instructions, etc. carried by the signals) to the working memory 775, from which the processor(s) 710 retrieves and executes the instructions. The instructions received by the working memory 775 may optionally be stored on a non-transitory storage device 725 either before or after execution by the processor(s) 710.

It should further be understood that the components of computer system 700 can be distributed across a network. For example, some processing may be performed in one location using a first processor while other processing may be performed by another processor remote from the first processor. Other components of computer system 700 may be similarly distributed. As such, computer system 700 may be interpreted as a distributed computing system that performs processing in multiple locations. In some instances, computer system 700 may be interpreted as a single computing device, such as a distinct laptop, desktop computer, or the like, depending on the context.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A medical device, comprising:
a housing that includes a mouthpiece;
a transducer coupled to the housing, the transducer being configured to generate a signal corresponding to one or more sound waves generated by air flowing towards the mouthpiece, the one or more sound waves being within an ultrasonic frequency range, and the air including medication for delivery to a user of the medical device;
and
circuitry coupled to the transducer, the circuitry being configured to determine, based on the signal, a property corresponding to the air flowing towards the mouthpiece.

2. The medical device of claim 1, wherein the ultrasonic frequency range does not include frequencies below twenty kilohertz.

3. The medical device of claim 1, wherein the one or more sound waves are within at least a one kilohertz frequency range.

4. The medical device of claim 1, wherein the medical device includes a physical feature configured to generate the one or more sound waves in response to the air flowing towards the mouthpiece.

5. The medical device of claim 4, wherein the physical feature is configured to divert a portion of the air flowing towards the mouthpiece.

6. The medical device of claim 5, wherein the physical feature includes a knife edge configured to divert the portion of the air flowing towards the mouthpiece in response to the air contacting the knife edge.

7. The medical device of claim 4, wherein the physical feature is configured to substantially generate the one or more sound waves when the air flows towards the mouthpiece and not to generate the one or more sound waves when the air flows away from the mouthpiece.

8. The medical device of claim 4, wherein the physical feature is configured to generate the one or more sound waves outside of the ultrasonic frequency range.

9. The medical device of claim 8, wherein the circuitry is configured to isolate the one or more sound waves within the ultrasonic frequency range from the one or more sound wave outside of the ultrasonic frequency range.

10. The medical device of claim 1, wherein the housing is configured enable the air to flow towards the mouthpiece in response to the user inhaling the air via the mouthpiece.

11. The medical device of claim 1, wherein the device is configured to induce the air to flow towards the mouthpiece by pushing the air using positive pressure.

12. The medical device of claim 1, further comprising a switch;
wherein the medical device is configured to introduce the medication into the air in response to an actuation of the switch; and
wherein the circuitry is configured to determine the property in response to the actuation of the switch.

13. The medical device of claim 1, wherein the transducer is configured to generate a respective signal corresponding to the one or more sound waves generated in response to air flowing towards and away from the mouthpiece.

14. The medical device of claim 13, wherein the circuitry is configured to determine, based on the respective signals corresponding to the one or more sound waves generated in response to air flowing, respectively, towards and away from the mouthpiece, respective properties corresponding to the air flowing towards and away from the mouthpiece.

15. The medical device of claim 1, wherein the property includes a rate of flow of the air flowing towards the mouthpiece or a volume of the air flowing towards the mouthpiece.

16. The medical device of claim 1, further comprising a medicine receptacle, wherein the medicine included within the air flowing towards the mouthpiece is inserted into the medical device via the medicine receptacle.

17. The medical device of claim 1, further comprising:
a wireless interface coupled to the circuitry; and
wherein the circuitry is further configured to transmit, via the wireless interface, data indicative of the property corresponding to the air flowing towards the mouthpiece.

18. The medical device of claim 17, wherein the data indicative of the property includes data indicative of a change of the property over time.

19. A method, comprising:
determining, by circuitry and based on a signal corresponding to the one or more sound waves generated by air flowing towards a mouthpiece within a medical device, a property corresponding to the air flowing towards the mouthpiece included within the medical device;
wherein the one or more sound waves are within an ultrasonic frequency range and the air includes medication for delivery to a user of the medical device.

20. The method of claim 19, wherein the ultrasonic frequency range does not include frequencies below twenty kilohertz.

21. The method of claim 19, wherein the one or more sound waves are within at least a one kilohertz frequency range.

22. The method of claim 19, wherein the one or more sound waves is generated by a physical feature of the medical device in response to the air flowing towards the mouthpiece.

23. The method of claim 22, wherein a portion of the air flowing towards the mouthpiece is diverted by the physical feature.

24. The method of claim 23, wherein the physical feature includes a knife edge;
wherein the portion of the air flowing towards the mouthpiece is directed by the knife edge in response to the air contacting the knife edge.

25. The method of claim 22, wherein the one or more sound waves are substantially generated when the air flows towards the mouthpiece; and
wherein the one or more sound waves are not generated when the air flows away from the mouthpiece.

26. The method of claim 22, wherein the one or more sound waves are generated outside of the ultrasonic frequency range.

27. The method of claim 26, further comprising:
isolating, by the circuitry, the one or more sound waves within the ultrasonic frequency range from the one or more sound wave outside of the ultrasonic frequency range.

28. The method of claim 19, wherein the air is enabled to flow towards the mouthpiece in response to the user inhaling the air via the mouthpiece.

29. The method of claim 19, wherein the air is induced to flow towards the mouthpiece by a positive pressure.

30. The method of claim 19, further comprising:
determining, by the circuitry, the property in response to an actuation of a switch included in the medical device;
wherein the medication is introduced into the air in response to the actuation of the switch.

31. The method of claim 19, wherein a respective signal corresponding to the one or more sound waves is generated in response to air flowing, respectively, towards and away from the mouthpiece.

32. The method of claim 31, further comprising: determining, by the circuitry and based on the respective signals corresponding to the one or more sound waves generated in response to air flowing, respectively, towards and away from the mouthpiece, respective properties corresponding to the air flowing towards and away from the mouthpiece.

33. The method of claim 19, wherein the property includes a rate of flow of the air flowing towards the mouthpiece or a volume of the air flowing towards the mouthpiece.

34. The method of claim 19, wherein the medical device further comprises a medicine receptacle; and
wherein the medicine included within the air flowing towards the mouthpiece is inserted into the medical device via the medicine receptacle.

35. The method of claim 19, wherein the medical device further comprises a wireless interface coupled to the circuitry; wherein the method further comprises: transmitting, via the wireless interface and by the circuitry, data indicative of the property corresponding to the air flowing towards the mouthpiece.

36. The method of claim 35, wherein the data indicative of the property includes data indicative of a change of the property over time.

37. An medical apparatus, comprising:
a means for generating a signal corresponding to one or more sound waves generated by air flowing towards a mouthpiece of a medical apparatus, wherein the one or more sound waves are within an ultrasonic frequency range and the air includes medication for delivery to a user of the medical apparatus; and
a means for determining, based on the signal, a property corresponding to the air flowing towards the mouthpiece.

38. The medical apparatus of claim 37, further comprising:
a means for generating the one or more sound waves in response to the air flowing towards the mouthpiece.

39. The medical apparatus of claim 38, wherein the means for generating the one or more sound waves in response to the air flowing towards the mouthpiece comprises: a means for diverting a portion of the air flowing towards the mouthpiece.

40. The medical apparatus of claim 38 wherein the means for generating the one or more sound waves in response to the air flowing towards the mouthpiece comprises: a means for generating the one or more sound waves outside of the ultrasonic frequency range.

41. The medical apparatus of claim 40, further comprising a means for isolating the one or more sound waves within the ultrasonic frequency range from the one or more sound wave outside of the ultrasonic frequency range.

42. The medical apparatus of claim 37, further comprising a means for enabling the air to flow towards the mouthpiece in response to the user inhaling the air via the mouthpiece, or for inducing the air to flow towards the mouthpiece using positive pressure.

43. The medical apparatus of claim 37, further comprising:
a means for triggering an introduction of the medication into the air; and
a means for determining the property in response to an actuation of the means for triggering the introduction of the medication into the air.

44. The medical apparatus of claim 37, further comprising:
a means for generating a respective signal corresponding to the one or more sound waves generated in response to air flowing, respectively, towards and away from the mouthpiece; and
a means for determining, based on the respective signals, respective properties corresponding to the air flowing, respectively, towards and away from the mouthpiece.

45. The medical apparatus of claim 37, further comprising:
a means for transmitting, wirelessly, data indicative of the property corresponding to the air flowing towards the mouthpiece;
wherein the data indicative of the property includes data indicative of a change of the property over time.
